(19) United States
(12) Patent Application Publication
(10) Pub. No.: US 2017/0014201 A1
(43) Pub. Date: Jan. 19, 2017

(54) Non-Invasive Head Stabilization System

(71) Applicant: Joachim A. Grotenhuis, Arnhem (NL)

(72) Inventors: Joachim A. Grotenhuis, Arnhem (NL); Libbe Jonkman, Ureterp (NL)

(21) Appl. No.: 15/207,739

(22) Filed: Jul. 12, 2016

Related U.S. Application Data
(60) Provisional application No. 62/191,642, filed on Jul. 13, 2015.

(51) Int. Cl. A61B 90/14; A61G 13/12

(52) U.S. Cl. CPC A61B 90/14; A61G 13/121; A61G 13/127; A61G 2203/10

(57) ABSTRACT

A head fixation device and method of stabilizing a head of a patient includes a base, one or more clamp members, and an actuator connected with each of the one or more clamp members. The base supports the head of the patient resting thereon. The one or more clamp members extend from the base and each selectively move from an adjustable position to a fixed position in response to movement of the actuators. The one or more clamp members provide the ability to conform to the contour of the patient's head to provide an area of contact for stabilization. The one or more clamp members also provide the ability to provide a point of contact for stabilization with an attached tip on each of the one or more clamp members.

### PRIORITY

This application claims priority to U.S. Provisional Patent Application Ser. No. 62/191,642, filed Jul. 13, 2015, entitled “Non-invasive Intraoperative Skull Stabilization System Specifically for Children,” the disclosure of which is incorporated by reference herein.

### BACKGROUND

The systems and methods disclosed pertain to the field of patient stabilization, and in particular head and neck stabilization using head fixation devices (hereinafter referred to as “HFDs” or “HFD” in singular).

HFDs are sometimes used during a variety of surgical and other medical procedures, for example during head or neck surgery or testing where it would be desirable to securely support a patient's head in a certain position. The HFDs described herein are, in some versions, particularly suitable for use with patients having weak cranial bone structure and/or smaller head size, e.g., pediatric patients. However, in some versions the HFDs described herein may also be used or adapted for use with patients having normal or fully developed cranial bone structure and/or head size.

While a variety of HFDs have been made and used, it is believed that no one prior to the inventor(s) has made or used the devices, systems, and methods as described herein. Other aspects, features, and techniques within the scope of the present disclosure will become more apparent to those of ordinary skill in the art from the following description taken in conjunction with the drawings.

### BRIEF DESCRIPTION OF THE DRAWINGS

FIG. 1 depicts a perspective view of an exemplary HFD.

FIG. 2 depicts a perspective view of another exemplary HFD.

FIG. 3 depicts a perspective view of the base of the HFD of FIG. 2.

FIG. 4 depicts a bottom view of the of the base of FIG. 3.

FIG. 5 depicts a side elevation view of the base of FIG. 3.

FIG. 6 depicts a perspective view of the clamp member of the HFD of FIG. 2.

FIG. 7 depicts a partially exploded perspective view of the clamp member of FIG. 6.

FIG. 8 depicts a perspective view of the yoke and actuator of FIG. 7.

FIG. 9 depicts a front elevation view of the yoke and actuator of FIG. 7.

FIG. 10 depicts a cross-sectional view of the yoke and actuator of FIG. 7 taken along section line 10-10 of FIG. 9.

FIG. 11 depicts a side elevation view of the arm of FIG. 7.

FIG. 12 depicts an exploded side elevation view of the arm of FIG. 7.

FIG. 13 depicts a side elevation view of a proximal link of the arm of FIG. 7.

FIG. 14 depicts a perspective view of the proximal link of FIG. 13.

FIG. 15 depicts a side elevation view of an intermediate link of the arm of FIG. 7.

FIG. 16 depicts a perspective view of the intermediate link of FIG. 15.

FIG. 17 depicts a side elevation view of a distal link of the arm of FIG. 7.

FIG. 18 depicts a perspective view of the distal link of FIG. 17.

FIG. 19 depicts a side elevation view of the atraumatic tip of the arm of FIG. 7.

FIG. 20 depicts a perspective view of the atraumatic tip of FIG. 19.

FIG. 21 depicts a perspective view of another exemplary HFD having a thermal control system.

FIG. 22 depicts a cross-sectional view of the arm of the HFD of FIG. 21 taken along section line 22-22 of FIG. 21.

FIG. 23 depicts a perspective view of another exemplary configuration of the HFD of FIG. 1.

FIG. 24 depicts a perspective view of another exemplary configuration of the HFD of FIG. 1.

FIG. 25 depicts a perspective view of another exemplary configuration of the HFD of FIG. 1.

FIG. 26 depicts a perspective view of another exemplary base that can be used interchangeably with the HFDs described herein.

FIG. 27 depicts a side elevation view of an exemplary head pad that can be used interchangeably with the HFDs described herein.

The drawings are not intended to be limiting in any way, and it is contemplated that various embodiments of the present disclosure may be carried out in a variety of other ways, including those not necessarily depicted in the drawings. The accompanying drawings incorporated in and forming a part of the specification illustrate several aspects, and together with the description serve to explain the principles of the present disclosure; it being understood, however, that the scope of the present disclosure is not limited to the precise arrangements shown.

### DETAILED DESCRIPTION

The following description of certain embodiments should not be used to limit the scope of the present disclosure. Other examples, features, aspects, embodiments, and advantages will become apparent to those skilled in the art from the following description. As will be realized, various aspects of the present disclosure may take alternate forms, or have alternate or additional embodiments, without departing from the scope of the present disclosure. Accordingly, the drawings and descriptions should be regarded as illustrative in nature and not restrictive.

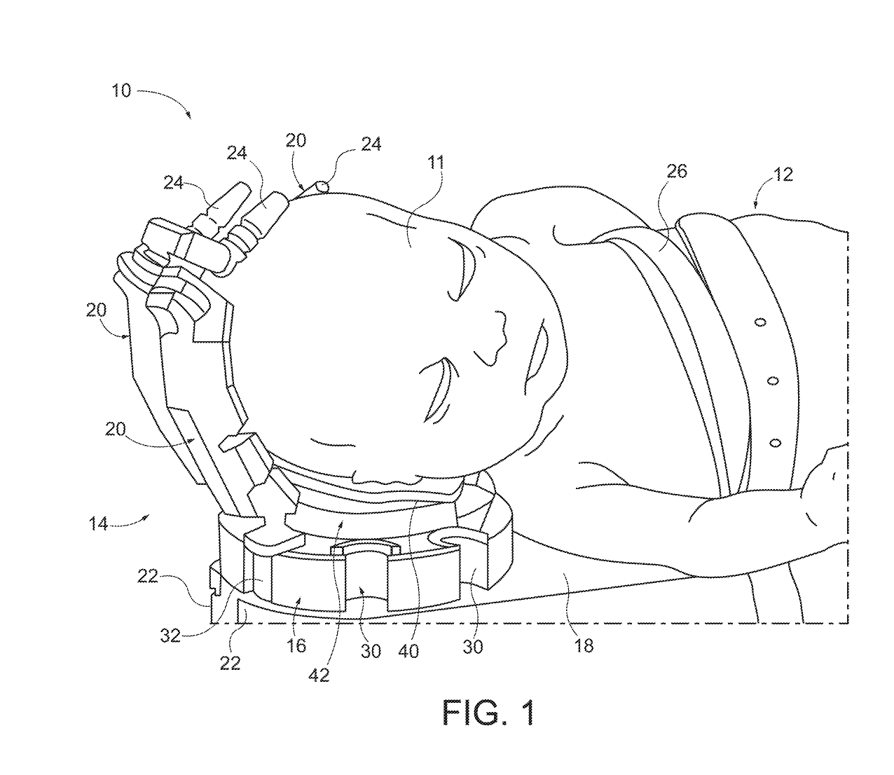

An exemplary patient stabilization system (10) as shown in FIG. 1 supports a head (11) of a patient, such as a child (12). The patient stabilization system (10) includes an HFD (14) removably mounted to a table (18). The HFD (14) includes a base (16), one or more clamp members (20) extending distally from the base (16), and one or more actuators (22) that connect with respective clamp members (20). In the present example, the base (16) is removably attached to the table (18). In other examples the base (16) removably attaches to the table (18) indirectly, e.g. by connecting with one or more adapters, which attach with the table (18). The base (16) of the present example is configured to support the head (11) of the child (12) resting thereon such that at least a portion of the weight of the head (11) is supported by the base (16).

In conjunction with the base (16), each of the clamp members (20) is selectively adjustable to contact that patient's head (11) to stabilize the head (11). Each of the actuators (22) is selectively movable between an unlocked position and a locked position. When the actuator (22) is in the unlocked position, the clamp member (20) is in an adjustable state as will be described further below. When the actuator (22) is in the locked position, the clamp member (20) is in a fixed state. As will be apparent to those of ordinary skill in the art in view of the teachings herein, the fixed state of the clamp member (20) is not required to be the same in all applications. For instance, one fixed state for the clamp member (20) may have a certain curvature of the clamp member (20), while another fixed state for the same or different clamp member (20) can have more or less curvature. Each clamp member (20) is configured to at least partially engage a portion of the head (11) when in the fixed state to contribute to at least partially stabilizing the head (11). Thereby, collectively the base (16) and multiple clamp members (20) stabilize the head (11) in order to immobilize the head (11) relative to the table (18) for performing a medical procedure. In the manner described above, the HFD (14) can be described as having a palm and fingers configuration that supports the head (11) of a patient (12).

As described herein, the term “proximal” generally refers to a location proximate to the base (16), whereas the term “distal” generally refers to a location extending upward from the base (16) over the head (11) of the child (12). The terms “proximal” and “distal” are merely relative positional terms and not intended to limit the invention described herein. Similarly, the term “longitudinal” generally refers to a direction extending between proximal and distal locations, whereas “transverse” generally refers to a direction perpendicular to the longitudinal direction. The patient may be the child (12) as shown herein, but it will be appreciated that the HFD (14) may be used with any patient, such as a pediatric patient, an adult patient, or other patient with or without weak cranial bone structure.

In the present example of FIG. 1, three clamp members (20) are positioned about the base (16), which is annular and generally planar, and extend therefrom to engage the head (11) for stabilization. While three clamp members (20) may be used, a greater or fewer number of clamp members (20) may be connected to the base (16) to provide the degree of stability desired. In one version, up to eight clamp members (20) may be used with base (16) to stabilize the head (11). In other versions, three to five clamp members (20) may be used with the base (16).

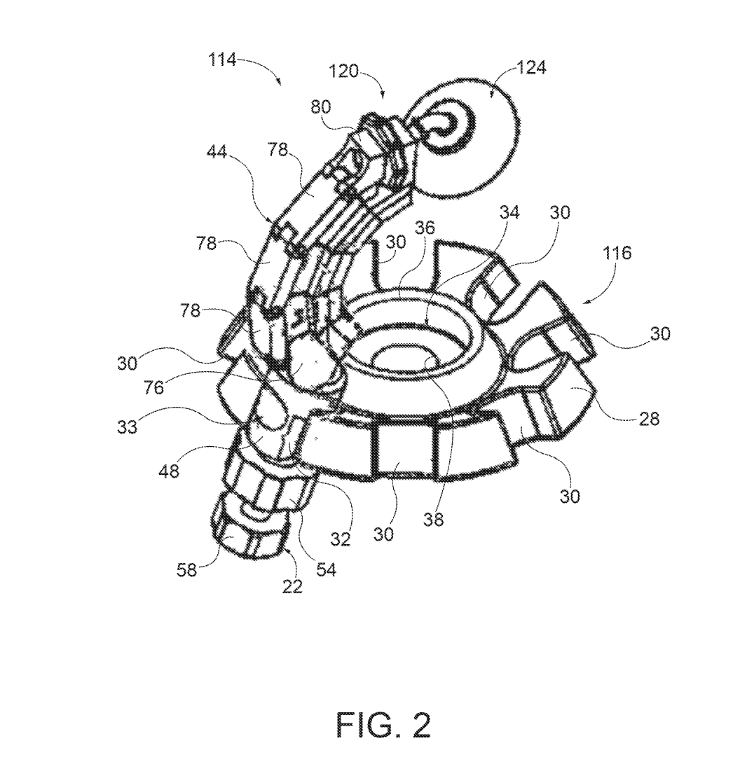

FIG. 1 also illustrates the clamp member (20) having an elongate atraumatic tip (24) configured to directly engage the head (11) for stabilization. Elongate atraumatic tip (24) distally tapers toward head (11) and is more elongate in the distal direction than the transverse direction. In some versions, elongate atraumatic tip (24) is configured such that it has the size and shape of a human fingertip that is operable to support the head (11). In other words, the elongate atraumatic tip (24) is distally elongated to engage the head (11), but relatively narrows so as to provide clearance around the elongate atraumatic tip (24) for the medical professional to access the head (11) during the medical procedure. However, it will be appreciated that alternative tips configured to provide sufficient stability and clearance for performing the medical procedure may also be used. For example, FIG. 2 shows a clamp member (120) with a cup atraumatic tip (124) that is generally circular in order to provide stability to the head (11). To this end, the following description provides additional details regarding the cup atraumatic tip (124), but it will be appreciated that any such tip configured to provide desirable stability and clearance may be used. Additionally, elongate atraumatic tip (24) and cup atraumatic tip (124) may be used interchangeably with either clamp member (20) and/or clamp member (120).

The term “atraumatic” used herein refers to one or more structural feature that reduce the likelihood of tissue damage while engaged with the tissue. Atraumatic tips (24, 124) thus engage the head (11) without damaging the engaged tissue of the head (11). While some skin abrasions and/or bruising may occur with such “atraumatic” engagement, it will be appreciated that the atraumatic tips (24, 124) inhibit skin breakage and do not attach directly to the skull within the head (11). However, alternative configurations may use clamp members that are fitted with invasive tips configured to attach directly to the skull. The HFD (14) is thus not intended to be unnecessarily limited to clamp members with atraumatic engagement. It will be further appreciated that as used herein, the term “non-invasive” may also be used interchangeably with “atraumatic.”

While the HFD (14) is configured to support and stabilize the head (11) of the child (12), the patient stabilization system (10) may further include additional movement restriction devices for stabilizing the remainder of the child (12). For example, a strap (26) is shown in FIG. 1 wrapped around both the child (12) and the table (18) to further stabilize the child (12) during the medical procedure. Alternative and/or additional movement restriction devices may be used with the HFD (14) as desired by the medical professional.

FIG. 2 illustrates an HFD (114) including the clamp member (120) and a base (116).

The base (116) is generally similar to the base (16), but with a distally upturned annular flange (28) for mounting the clamp member (120) to extend inward over the base (116) and toward the head (11). To this end, like numbers indicate like features. Of course, the clamp members (20, 120) may be mounted to any of the bases (16, 116), and in any arrangement configured for engagement between the clamp members (20, 120) and the head (11).

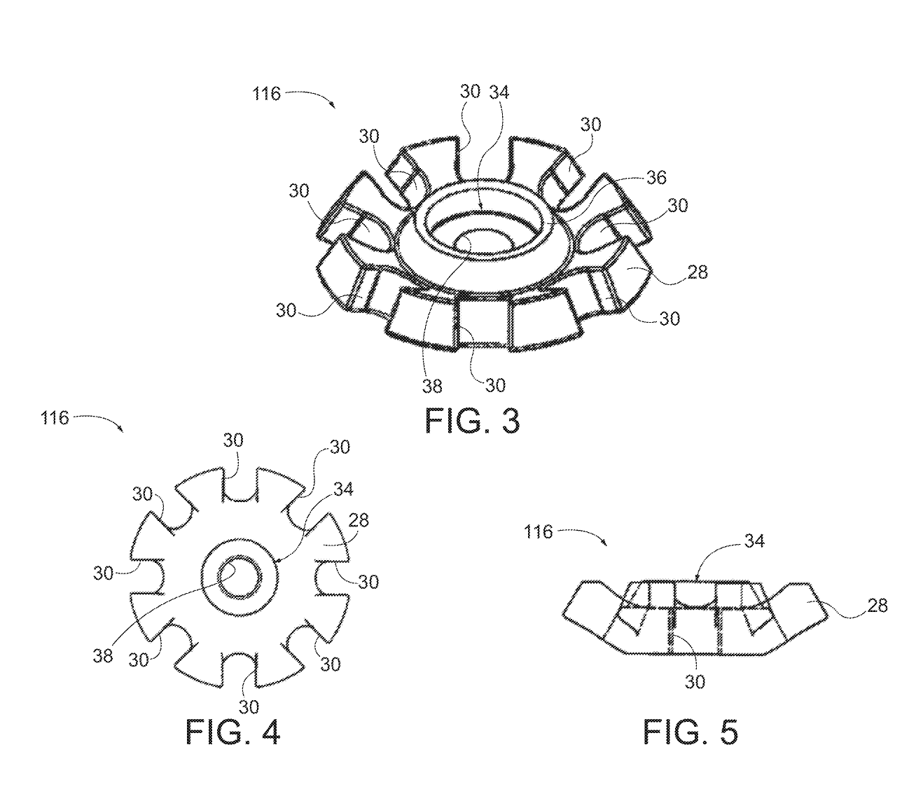

With respect to FIGS. 2-5, the annular flange (28) has a plurality of grooves (30) radially positioned about the annular flange (28) and configured to removably receive the clamp members (120). Each groove (30) extends through the annular flange (28) from a distal face of the annular flange (28) to a proximal face of the annular flange (28). The clamp member (120) further includes a tongue (32) configured to be slidably received within each groove (30). Thereby, the tongue (32) of the clamp member (120) is removably received within any of the grooves (30) for radial positioning about the annular flange (28). In the present example, the annular flange (28) includes eight grooves (30) equiangularly positioned about the annular flange (28) such that the clamp members (120) may be positioned and repositioned as desired in any one of the grooves (30). It will be appreciated that alternative coupling arrangements and numbers thereof for removably connecting the clamp member (120) to the base (116) may be used. The invention is thus not intended to be unnecessarily limited to the tongue (32) and groove (30) coupling arrangement shown and described herein.

The base (116) further includes a central portion (34) surrounded by the annular flange (28) that includes a hollow, frustoconical head support collar (36) and a bore (38) extending therethrough. The bore (38) and the hollow head support collar (36) may be configured to receive a mounting clamp (not shown) therein for removably and rigidly mounting the base (116) to the table (18) or other adapter that may connect with the table (18). In addition to, or as an alternative to, the base (116), other base units and swivel adapters may be useful for such mounting and will be apparent to those of ordinary skill in the art in view of the teachings herein.

The head support collar (36) extends distally from the bore (38) to thereby provide clearance for the mounting clamp (not shown) underneath while also receiving a head pad (40) (see FIGS. 1 and 27) thereon to collectively define a head pedestal (42) (see FIG. 1). The head pedestal (42) is thus configured to receive the head (11) such that the head (11) is comfortably supported from beneath while stabilized via clamp members (120). The head pad (40) can be gel pads in some versions, foam pads in some versions, rubber pads in some versions, or any other type of pad suitable to contact and support a patient's head and/or face and/or neck. Furthermore, while the present example of FIG. 1 illustrates round or circular shaped pads, other shapes may be used and can include, for example and not by way of limitation, horseshoe, straight, triangle, or curved, among other shapes. The head pad (40) shown with the base (16), is also usable with the bases (116, 216). In one version, the head pad (40) is configured to provide support to both the head (11) and neck of the patient. In such versions, the head pad (40) can have a non-uniform profile so as to accommodate the natural contour of the patient's head and neck (see FIG. 27). In other words, the head pad (40) can be configured with a profile that matches or closely matches the contour of the patient's head and neck to provide support and comfort.

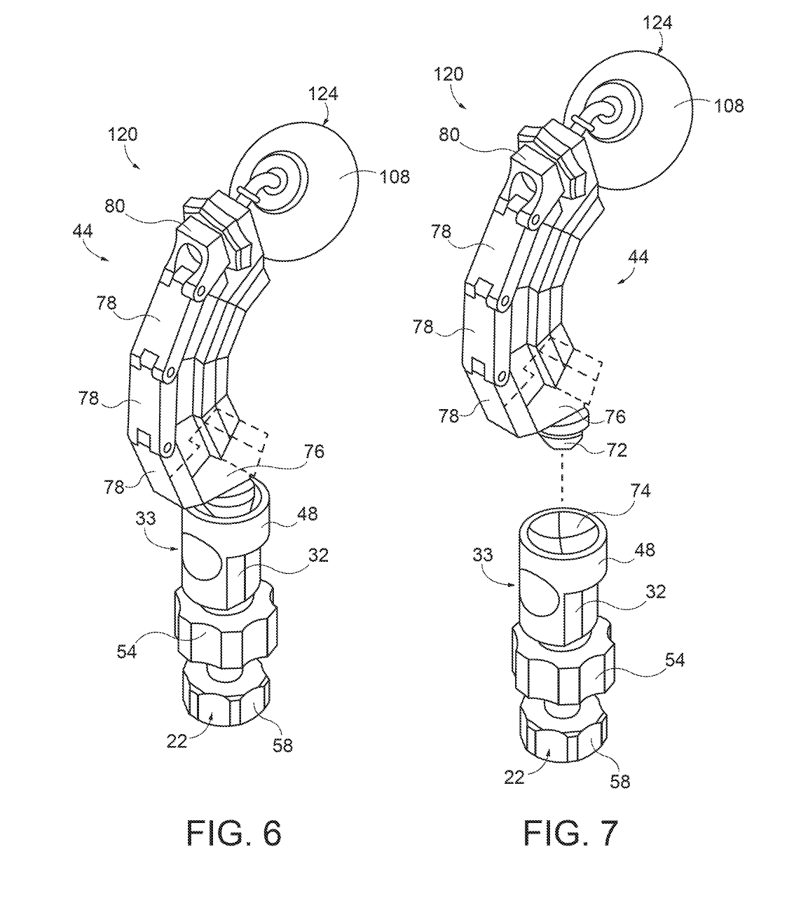

With respect to FIGS. 6-7, the clamp member (120) includes a yoke (33), an arm (44), which extends distally from the yoke (33), and the actuator (22). In the present example, the actuator (22) is secured to the yoke (33) and connected to the arm (44) therethrough as described further below. Furthermore, each arm (44) of the present example is operatively connected to a respective actuator (22) such that each arm (44) may be adjusted independently of the other arms (44) in use for stabilizing the head (11) (see FIG. 1). An alternative actuator (not shown) may be operatively connected to two or more arms (44) in use for simultaneously adjusting two or more arms (44).

FIGS. 7-10 show the yoke (33) and the actuator (22) in greater detail. The yoke (33) includes a yoke body (48), which defines the tongue (32) described above, and a yoke coupler (50). The yoke coupler (50) is coaxially received within the yoke body (48) such that each extends along a longitudinal axis. A pin (52) extends through the yoke body (48) and the yoke coupler (50) to secure the yoke coupler (50) within the yoke body (48). Furthermore, a yoke knob (54) is connected to a proximal portion of the yoke coupler (50) and configured to provide a surface for gripping while adjusting the arm (44). The yoke body (48) includes a proximal surface that is configured to abut the distal surface of the base (116) when the tongue (32) is positioned within the groove (30) of the base (116). Also, the distal surface of the yoke knob (54) is configured to abut the proximal surface of the base (116) when the tongue (32) is positioned within the groove (30) of the base (116). In this manner, with the tongue (32) within the groove (30), and the contact between the base (116) and the respective yoke body (48) and yoke knob (54), the clamp member (20) is able to be retained and/or secured with the base (116). Various ways to securely and selectively connect the clamp member (120) with the base (116) will be apparent to those of ordinary skill in the art in view of the teachings herein. For instance, in one version, clamp member (120) may be configured such that the yoke knob (54) may translate along the yoke coupler (50) to tighten and/or loosen the connection between the clamp member (120) and the base (116). For example, yoke knob (54) may be threadably connected with the yoke coupler (50) to make such adjustments. In another version, the actuator (22), the yoke body (48), and yoke coupler (50) may move together and translate relative to the yoke knob (54), e.g. by a threaded connection, in response to movement of the actuator (22) to thereby tighten and/or loosen the connection between the clamp member (120) and the base (116).

In the present example, the actuator (22) includes a cable (56) and a rotatable actuator knob (58). Pulling the cable (56) proximally is configured to move the arm (44) such that it bends or flexes inward toward a central longitudinal axis defined by the base (116). In other words, pulling the cable (56) proximally is configured to move the arm (44) such that it bends or flexes inward toward the head (11) of a patient (12) supported on the base (116). In the present example, the actuator knob (58) is configured to be selectively rotated for pulling and/or releasing the cable (56) to thereby move the clamp member (120) to or from the adjustable state to or from the fixed state. Other ways to selectively pull the cable (56) to selectively move the clamp member (120) will be apparent to those of ordinary skill in the art in view of the teachings herein. For instance, in some versions the user may manipulate the arm (44) to achieve a desired contact pattern with the head (11), this action moves the cable (56) or sets the cable (56) such that the actuator (22) can then be rotated to retain the cable (56) such that the clamp member (120) assumes its fixed state.

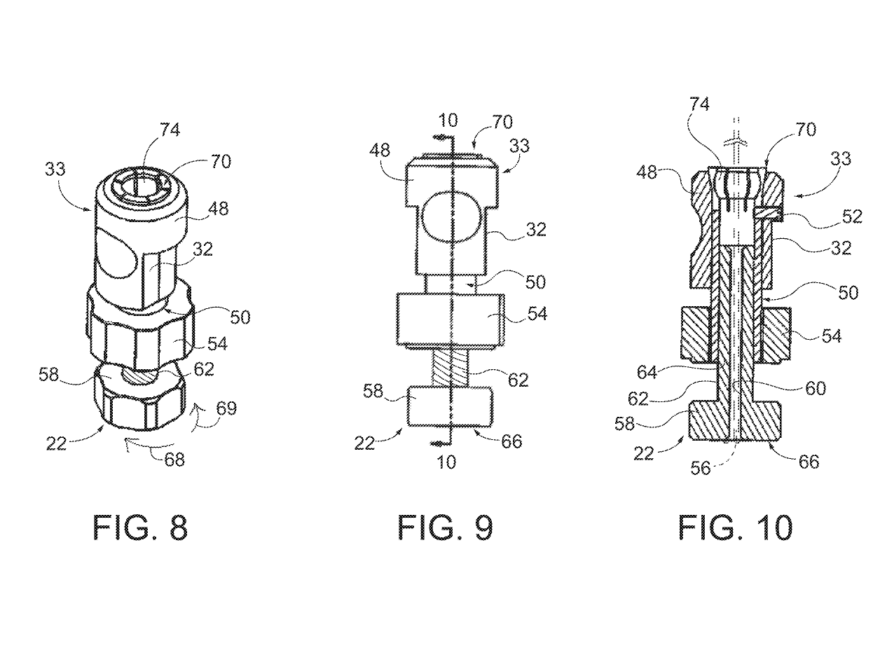

As best shown in FIGS. 8-10, the actuator (22) defines a proximal cable conduit (60) extending longitudinally therethrough and the actuator (22) includes an outer threading (62) that cooperatively engages an inner threading (64) within the yoke coupling (50). Furthermore, the cable (56) extends proximally from the arm (44) through the proximal cable conduit (60) and to a proximal end portion (66) of the actuator knob (58) where the cable (56) is affixed to the proximal end portion (66). Thereby, selectively rotating the actuator knob (58) clockwise, as indicated by arrow (68), causes the actuator knob (58) to move proximally relative to the yoke (33) and, in turn, proximally pull the cable (56) into tension during use. Of course, selectively rotating the actuator knob (58) counterclockwise, as indicated by arrow (69), causes the actuator knob (58) to move distally relative to the yoke (33) and, in turn, distally release tension in the cable (56) during use. In one example, the cable (56) is a thread having at least some resilience. However, the cable (56) may be any structure or structures configured to transmit force from the actuator knob (58) to the arm (44).

As shown in FIGS. 7, 10, and 11, a distal end portion (70) of the yoke coupler (50) is configured to be movably mounted to a proximal end portion of the arm (44) to allow for relative movement therebetween with at least one degree of freedom. The present example includes a yoke ball joint (72) at the proximal end portion of the arm (44) and a yoke socket joint (74) at the distal end portion (70) of the yoke coupler (50) for three degrees of relative freedom. More particularly, the yoke ball and socket joints (72, 74) cooperate such that the relative position of the arm (44) is adjustable with respect to pitch, yaw, and roll. While the yoke ball and socket joints (72, 74) are generally adjustable, in the present example, tensioning the cable (56) as described above is configured to place the yoke ball joint (72) into frictional engagement with the yoke socket joint (74). The actuator (22) of the present example is thereby also configured to effectively lock movement of the arm (44) relative to the yoke (33) while the arm (44) engages the head (11) for greater stabilization. In contrast, releasing tension on the cable (56) is configured to sufficiently reduce frictional engagement between the ball and yoke socket joints (72, 74) for further repositioning or adjusting the arm (44) as desired by the medical professional. With the configuration described above, movement of the cable (56) controls adjustment of the clamp member (120) both in terms of the arm (44) bend or flex toward the head (11) or central longitudinal axis defined by the base (116), and also in terms of the arm (44) angle or position relative to the yoke (33) or the base (116).

As discussed above, the cable (56) is affixed to the proximal end portion (66) of the actuator knob (58). In some versions, cable (56) may be removably affixed to the proximal end portion (66) and may be detached from the proximal end portion (66), repositioned relative to the proximal end portion (66), and reaffixed. The medical professional may thereby adjust tension in the cable (56) and set or alter the orientation of the clamp member (120) when at rest in the adjustable position. Still in other versions, the cable (56) is fixed within clamp members (20, 120).

Adjustment of the cable (56) position also affects the amount of pressure that the arm (44) applies to the head (11). For instance, pulling the cable (56) proximally to a further extent will cause the arm (44) to bend or flex more inwardly toward the head (11) that is supported by the base (116). Once the arm (44) contacts the head (11), such pulling on the cable (56) proximally to a further extent increases the pressure between the arm (44) and the head (11). Stated another way, once the arm (44) contacts the head (11), such pulling on the cable (56) proximally to a further extent increases the force that the arm (44) applies to the head (11). In the reverse manner, the pressure or force can be decreased by moving the cable (56) distally. Other ways to control the force that the arm (44) applies to the head (11) will be apparent to those of ordinary skill in the art in view of the teachings herein.

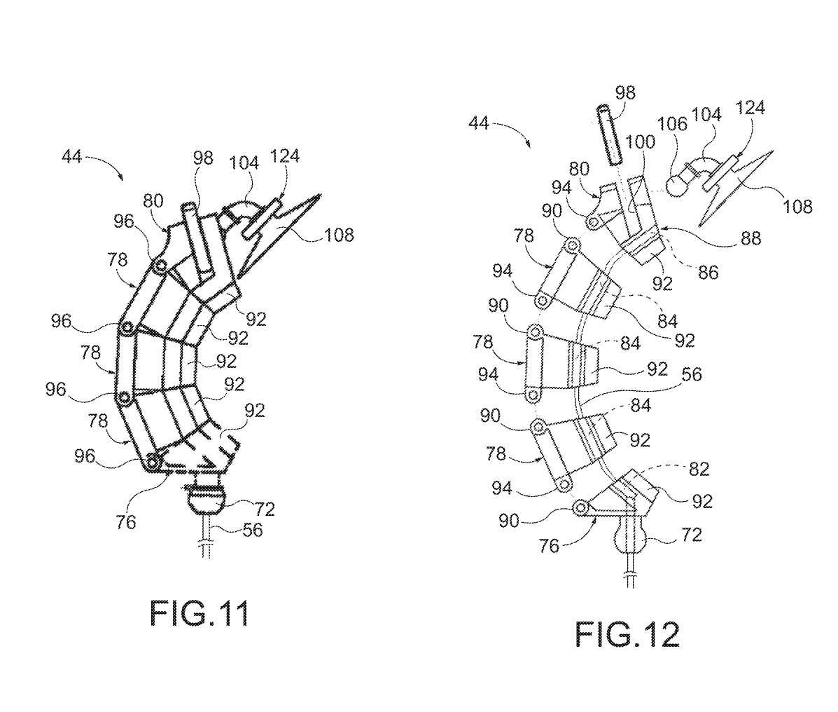

FIGS. 11-12 illustrate the arm (44) in greater detail as including the atraumatic tip (124) and a plurality of links (76, 78, 80) connected in series and extending proximally therefrom. Each link (76, 78, 80) is configured to articulate relative to any adjacent link (76, 78, 80) connected thereto so as to collectively adjust the arm (44) and define the overall contour of the arm (44). More particularly, the arm (44) includes a proximal link (76), a plurality of intermediate links (78), and a distal link (80). Each of the proximal, intermediate, and distal links (76, 78, 80) respectively includes a proximal, intermediate, and distal channel (82, 84, 86) extending respectively therethrough. The proximal, intermediate, and distal channels (82, 84, 86) align relative to each other with the overall contour of the arm (44) to collectively define a distal cable conduit (88) configured to receive a distal portion of the cable (56) projecting from the yoke (33). The cable (56) of the present example is affixed to the distal link (80) to direct articulation of each link (76, 78, 80) via the actuator (22) as discussed below in additional detail.

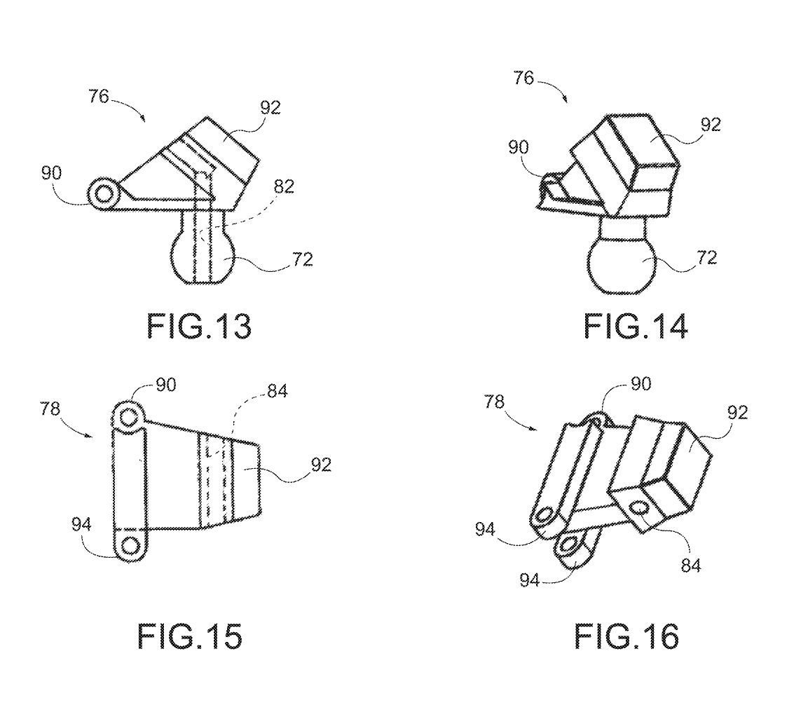

With respect to FIGS. 13-14, the proximal link (76) includes the yoke ball joint (72), the proximal channel (82), and a link coupling in the form of a tab ring (90). The yoke ball joint (72) extends proximally therefrom, and the proximal channel (82) is angled so as to extend from a distal face of the proximal link (76) and through the yoke ball joint (72) to provide a path for the cable (56) to be introduced into the yoke (33). An inner portion of the proximal link (76) further includes a support pad (92) that may be configured to engage the head (11). By way of example, the construction of support pad (92) may be the same or similar to that discussed above with respect to head pad (40). Opposite the inner portion, the outer portion of the proximal link (76) includes the tab ring (90) extending distally therefrom and configured to receive a portion of the intermediate link (78). Accordingly, the proximal channel (82) is generally positioned inward of the tab ring (90). As used herein the term “inner” refers to the portion of the arm (44) configured to be positioned adjacent to the head (11), whereas the term “outer” refers to the portion of the arm (44) configured to be positioned opposite the head (11). The terms “inner” and “outer” are thus used merely for positional reference and are not intended to limit the invention described herein.

The intermediate link (78) is shown in FIGS. 15-16 and includes the intermediate channel (84), the link coupling in the form of the tab ring (90), another link coupling in the form of a pair of offset rings (94), and the support pad (92). The support pad (92) is positioned at an inner portion of the intermediate link (78), whereas the offset rings (94) and the tab ring (90) are positioned at an outer portion of the intermediate link (78). More particularly, the tab ring (90) extends distally from the intermediate link (78) to be received between the pair of offset rings (94) of a distally adjacent link, such as the distal link (80) or another intermediate link (78). The pair of offset rings (94) extend proximally from the intermediate link (78) to receive the tab ring (90) of a proximally adjacent link, such as the proximal link (76) or another intermediate link (78). The rings (90, 94) are configured to receive a pin (96) (see FIG. 11) to pivotally connect the links (76, 78, 80) (see FIG. 11) for relative articulation. Notably, the pair of offset rings (94) on the intermediate link (78) are spaced apart from the tab ring (90) on the same intermediate link (78) a distance greater than the parallel distance along the support pad (92).

With the configuration of the links (76, 78, 80) described above, the series of links (76, 78, 80) accommodates greater articulation along the inner portion than the outer portion to move the arm (44) such that the arm (44) has a greater curvature along its inner surface compared to its outer surface. Furthermore, the links (76, 78, 80) have a maximum curvature that can be achieved, and this maximum curvature occurs when adjacent links contact each other on their respective inward portions. For instance, when the arm (44) is bent or flexed to its maximum curvature, the support pads (92) of adjacent links (76, 78. 80) will contact one another, as well as the surface of the links themselves, as shown in FIG. 11 for instance. In this way, the arm (44) is configured to provide a limit to the amount of force that can be exerted by the arm (44) onto the head (11) so as to not injure the head (11). In the opposite manner, the links (76, 78, 80) of the arm (44) can have less curvature in use such that adjacent links (76, 78, 80) do not contact one another along their inward most portions, and thus the support pads (92) of adjacent links (76, 78, 80) may not be in contact with one another in all configurations. In one example, arms (44) may be configured with more or their maximum curvature when used with a patient having a smaller head (11) compared to when used with a patient having a larger head (11). Other ways to configure the amount of curvature used with the arms (44) of the clamp member (120) depending on the application will be apparent to those of ordinary skill in the art in view of the teachings herein.

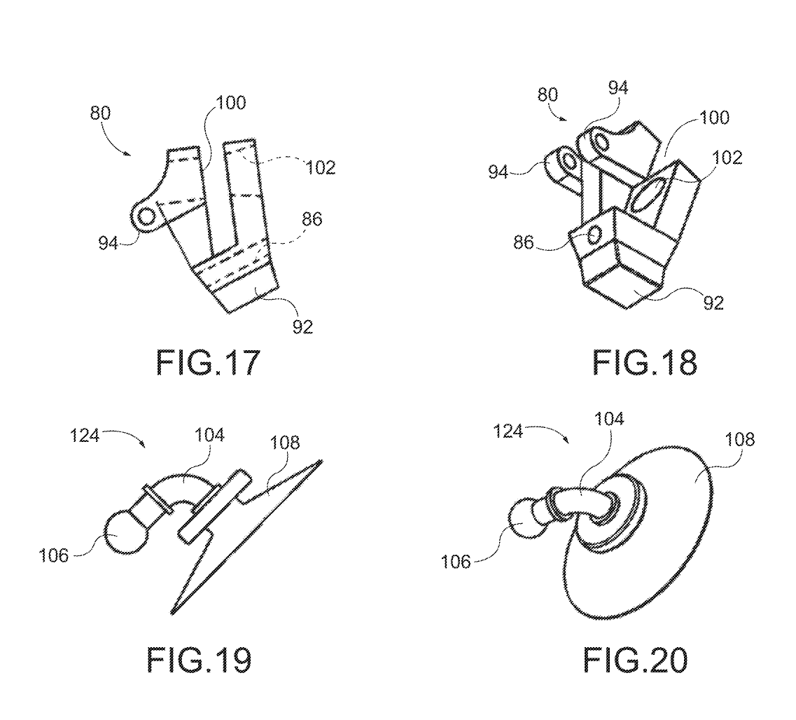

The distal link (80) is shown in FIGS. 17-18 and includes the distal channel (86), the link coupling in the form of the pair of offset rings (94) and the support pad (92). The support pad (92) is positioned at an inner portion of the distal link (80), whereas the offset rings (94) are positioned at an outer portion of the distal link (78). More particularly, the pair of offset rings (94) extend proximally from the distal link (80) to receive the tab ring (90) of one of the intermediate links (78) for pivotal connection with the pin (96). The distal link (80) further includes a tip control knob (98) (see FIG. 12) rotatably mounted within a slot (100). The tip control knob (98) connects to the cup atraumatic tip (124) through a distal hole (102), which receives the cup atraumatic tip (124) (see FIG. 19).

As described above, the clamp members (20, 120) are movable from an adjustable state to a fixed state based on movement of the cable (56). In the adjustable state, the links (76, 78 80) are operable for pivotal movement such that the curvature of the arms (44, 144) can be adjusted. In this manner, the pivoting action of the links (76, 78, 80) can be considered to cause the clamp members (20, 120) to move from a retracted state where the links (76, 78, 80) pivot toward one another to an extended state where the links (76, 78, 80) pivot away from one another. When the clamp members (20, 120) are in the adjustable state, the links (76, 78 80) may be configured in the extended state or the retracted state depending on the orientation of the pivoting links (76, 78, 80) that define the desired degree of curvature for the arms (44, 144) for a given stabilization application. When the clamp members (20, 120) are in the fixed state, the links (76, 78, 80) may also be configured in the extended state or the retracted state depending on the orientation of the pivoting links (76, 78, 80) that define the desired degree of curvature for the arm (44, 144) for a given stabilization application. Accordingly, the terms “adjustable state” and “fixed state” are not specific to any one position for the clamp members (20, 120) or any one shape or degree of curvature for the arms (44, 144) of the clamp members (20, 120).

The cup atraumatic tip (124) discussed briefly above is shown in FIGS. 19-20 in greater detail. In one example, the cup atraumatic tip (124) includes a stem, such as a curved elbow stem (104), extending between a stem ball joint (106) and a cup body (108). The stem ball joint (106) is received within a stem socket joint (not shown) within tip control knob (98) to allow for relative movement therebetween with at least one degree of freedom. The present example includes the stem ball joint (106) received within the stem socket joint (not shown) for three degrees of relative freedom. More particularly, the stem ball and socket joints (106, not shown) cooperate such that the position of the cup atraumatic tip (124) is adjustable relative to the distal link with respect to pitch, yaw, and roll for positioning on the head (11).

The cup body (108) of the present example is in the form of a cup pad configured to atraumatically engage the head (11). By way of example, the cup pad may be constructed of materials as those discussed above with respect to head pad (40) and/or the support pads (92). Alternatively, the cup body (108) may be relatively firm so as to more rigidly engage the head (11). In any case, the invention is not intended to be limited to the particular cup body (108) shown and described herein.

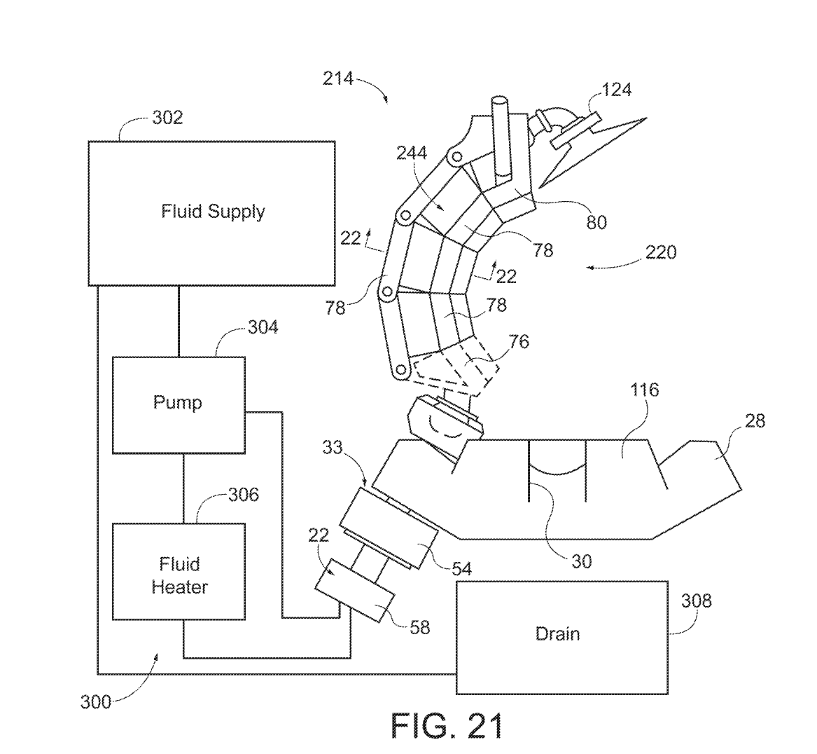

With respect to FIG. 21, an alternative HFD (214) includes a clamp member (220) operatively connected to a thermal control system (300) for warming the clamp member (220) during use. Clamp member (220) includes the yoke (33) received within the base (116), while the actuator (22) is operatively connected to a thermal arm (244). To this end, like numbers indicate like features described above in greater detail.

The thermal control system (300) includes a fluid supply (302), a pump (304), and a fluid heater (306). The pump (304) is configured to direct a fluid, such as water, from the fluid supply (302) and into the fluid heater (306). The fluid heater (306) heats the fluid to a desirable temperature as it flows therethrough and into the clamp member (220). The heated fluid flows distally toward the distal link (80), at which point the fluid proximally returns back to the pump (304) to be recirculated through the HFD (214). In order to flush the thermal control system (300), the pump (304) is reversed to pump the fluid back into the fluid supply (302) to be discarded to a drain (308).

As an alternative to or as an addition to the fluid heater (306), the thermal control system (300) may include a fluid cooler (not shown) configured to cool the fluid to the desirable temperature. By way of example, the fluid cooler (not shown) may be fluidly connected between the pump (304) and the clamp member (220) in parallel with the fluid heater (306). It will thus be appreciated that the thermal control system (300) is not intended to be unnecessarily limited to heating fluid, but may be configured for any thermal adjustments to the clamp member (220).

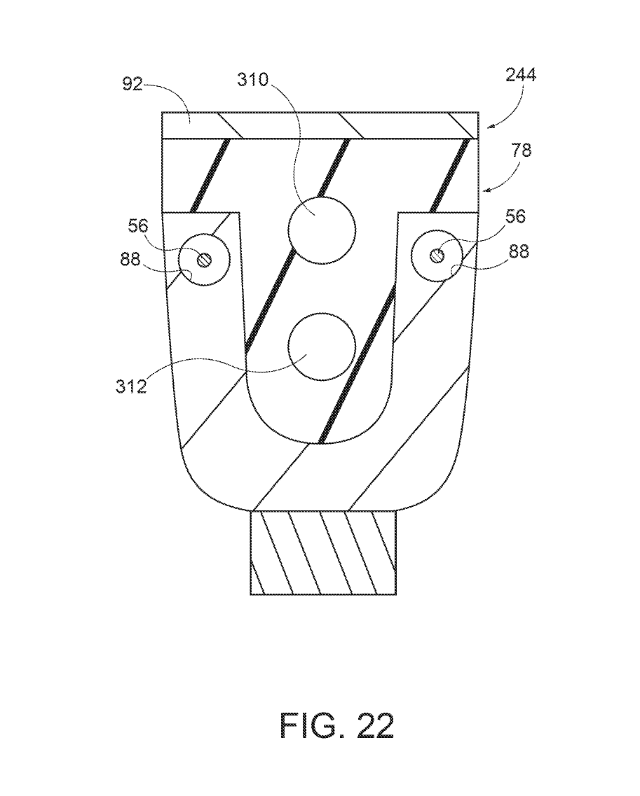

FIG. 22 shows one example of the thermal arm (244) in greater detail. The thermal arm (244) includes a delivery conduit (310) configured to receive heated fluid from the pump (304) and a return conduit (312) configured to direct the fluid back toward the pump (304) for recirculation as discussed above. In addition, the arm (244) has a pair of distal cable conduits (88) configured to receive a pair of cables (56), which generally operate as discussed herein with respect to the clamp member (120) (see FIGS. 10-12).

In use, with respect to FIGS. 1-12, the HFD (114) is mounted to the table (18), either directly or indirectly by way of one or more adapters, for stabilizing the head (11) of the patient (12). The medical professional rests the head (11) on the head pad (40) of the base (116) such that the head pad (40) supports at least a portion of the weight of the head (11) thereon. In some versions the base (116) and head pad (40) support at least a majority of the weight of the head (11) thereon. Neck support may also be provided by the base (116) and/or head pad (40). In order to stabilize the head (11), the medical professional positions the clamp member (120) such that one of the plurality of grooves (30) slidably receives the clamp member (120).

Generally, the clamp member (120) in the present example is freely adjustable prior to engagement with the head (11). The medical professional accordingly grips the arm (44) and positions the atraumatic cup tip (124) against the head (11). In one example, the medical professional manipulates the arm (44) with one or more of pitch, yaw, and roll relative to the head (11) to position the arm (44) in a desirable location. In addition, the medical professional may also manipulate the atraumatic cup tip (124) with one or more of pitch, yaw, and roll relative to the head (11) to position the atraumatic cup tip (124) in a desirable location. In positioning the arm (44), in some versions engagement between the clamp member (120) and the head (11) may be limited to the atraumatic cup tip (124). In other versions, the engagement between the clamp member (120) and the head (11) may be limited to one or more of the support pads (92) along the arm (44). Still in other versions, the engagement between the clamp member (120) and the head (11) may include both the atraumatic cup tip (124) as well as one or more of the support pads (92) along the arm (44).

With the head (11) and atraumatic cup tip (124) in position, the medical professional rotates the actuator knob (58) clockwise in order to rotatably drive the actuator knob (58) proximally relative to the yoke (33) through the threaded connection. Rotating the actuator knob (58) clockwise thus proximally pulls the cable (56) along the distal and proximal cable conduits (88, 60). As discussed above, this proximal movement of the cable (56) causes the arm (44) to bend or flex inward toward the head (11), while also placing the yoke ball joint (72) into frictional engagement with the yoke socket joint (74) such that the arm (44) adopts a fixed state. In this fixed state, the arm (44) is no longer freely adjustable relative to the yoke (33), or relative to the base (116) since the yoke (33) connects with the base as described above. Additionally, in this fixed state with the arm (44) contacting the patient's head (11), the arm (44) is no longer freely adjustable in the degree of curvature of the arm (44). If further adjustments of the arm (44) are needed, the medical professional may rotate the actuator knob (58) in the opposite direction to release tension on the cable (56), which reduces frictional engagement between the ball and yoke socket joints (72, 74) for further repositioning or adjusting the arm (44) as desired relative to the yoke (33) or base (116); and for repositioning or adjusting the curvature of the arm (44). In the event that greater stabilization of the head (11) is desirable to perform the medical procedure, the medical professional repeats the above steps with one or more clamp members (20, 120) until the collection of clamp members (20, 120) fully stabilizes the head (11) of the patient (12) for performing the medical procedure.

In one version, the tensioned cable (56) applies a torque about the pivotable link couplings (90, 94) and pins (96) to articulate the links (76, 78, 80), which, in turn, generally conform to the shape of the head (11). In such a version, the medical professional may continue to articulate the links (76, 78, 80) until the atraumatic cup tip (124) engages the head (11) and at least partially stabilizes the head (11).

After the medical procedure is completed, the medical professional unclamps the head (11) from the head pedestal (42) by rotating the actuator knob (58) counterclockwise and releasing the tension on the cable (56). In one example, the cable (56) is a pull cable such that distal movement of the cable (56) simply loosens the cable (56) and the arm is freely adjustable. In another example, the cable is a push-pull cable configured to transmit tension and compression. Accordingly, distal movement of the cable (56) directs the arm (44) away from the base (116) where there is some degree of separation between the links (76, 78, 80) for adjustability. In either case, the medical professional removes any remaining atraumatic tips (24, 124) from the head (11) in order to free the head (11) of the patient (12) from stabilization.

While the above description applies generally to the HFD (14, 114) shown in FIGS. 1-20, it will be appreciated that the HFD (214) of FIGS. 21-22 with the thermal control system (300) may be similarly operated. Also, as discussed above with respect to FIG. 1, the patient stabilization system (10) may be assembled with various clamp members (20, 120) in various configurations.

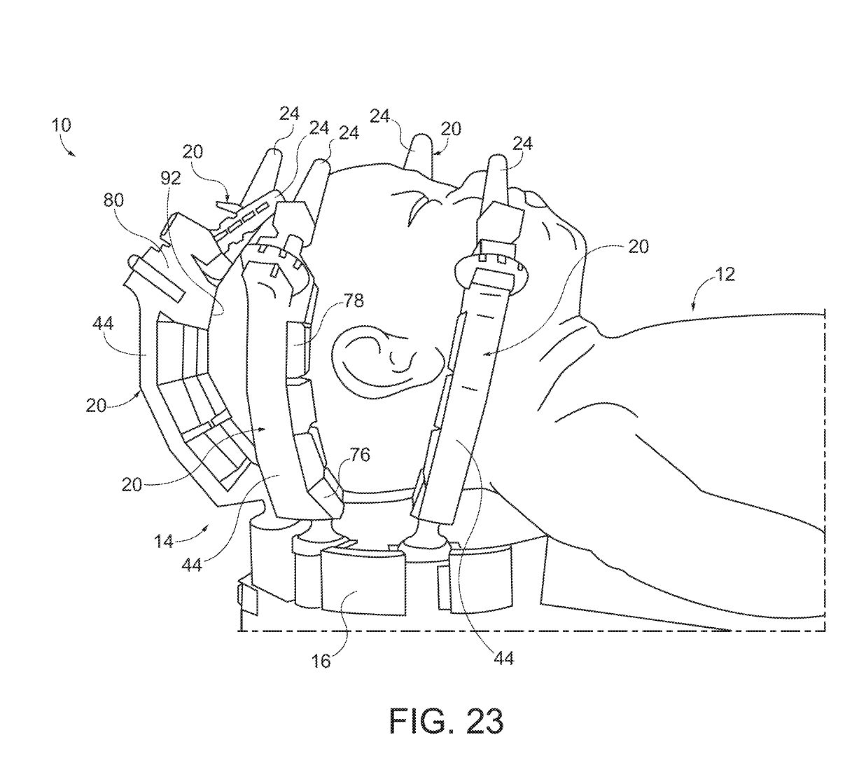

Alternative configurations for patient stabilization system (10) are illustrated in FIGS. 23-25, but it will be appreciated that other configurations for stabilization may be arranged as desired by the medical professional in view of the teachings herein. With respect to the discussion of FIGS. 23-25, while much of the description is directed to HFD (14) and clamp members (20), it should be understood that the description here applies equally to HFD (114) and clamp members (120).

FIG. 23 illustrates HFD (14) used in stabilizing the head (11) of a patient (12). In the present example, multiple clamp members (20) are used to achieve stabilization. More specifically five clamp members (20) are used. Also in the present example, elongated atraumatic tips (24) are used with each clamp member (20). However, in some other versions one or more of tips (24) may be replaced with cup atraumatic tips (124).

As shown in the present example of FIG. 23, at least one or more of arms (44) of clamp members (20) are configured such that all the support pads (92) of the plurality of links (76, 78, 80) contact the head (11) to provide stabilization support. For instance, the support pads (92) of the arms (44) of the clamp member (20) positioned along the top of the head (11) each contact the head (11). In this manner, instead of clamp member (20) creating a point of contact for stabilization, clamp members (20) are configured to provide an area of contact for stabilization. In this manner the area of contact for stabilization may be defined as the sum of the surface area of each support pad (92) contacting the head (11). With the curved finger-like shape of the arms (44), the area of contact for stabilization may be provided as a curved surface area. In this manner the area of stabilization provided by the arms (44) can follow the contour of the patient's head (11). Otherwise stated, the area of contact for stabilization can follow an arc length of the patient's head (11) along the longitudinal plane coinciding with the arm (44).

With some versions having this configuration with the area of contact, the arm (44) is adjusted in a way such that the arm (44) adopts a shape that closely matches, or is the same as, the contour of the patient's head (11) along the location where the arm (44) is positioned along the patient's head (11). In this way, the clamp member (20) is configured to provide continuous support along the contour of the patient's head (11). In some versions, this support may be continuous along the contour as mentioned, but in other versions this support may be discontinuous where one or more of the support pads (92) along the plurality of links (76, 78. 80) do not engage or contact the patient's head (11). Where the HFDs (14, 114) include multiple clamp members (20, 120), each clamp member (20, 120) is independently adjustable such that each clamp arm (20) may provide a point of stabilization, an area of stabilization as described above, or both point of stabilization at the tip (24, 124) and area of stabilization along the arm (44, 144), or any combination thereof. Moreover, wherever an area of stabilization is used, it may be either continuous or discontinuous as mentioned above.

With configurations that use the area of stabilization as compared to the point of stabilization, the HFD can provide a larger surface area of engagement between the support pads (92) of the arm (44) and the head (11). In some instances, the force required for stabilization is thus spread over a larger area, which can provide less trauma or risk of trauma to the engaged tissue and/or bone beneath the tissue. In this manner there is less pressure exerted on the head (11).

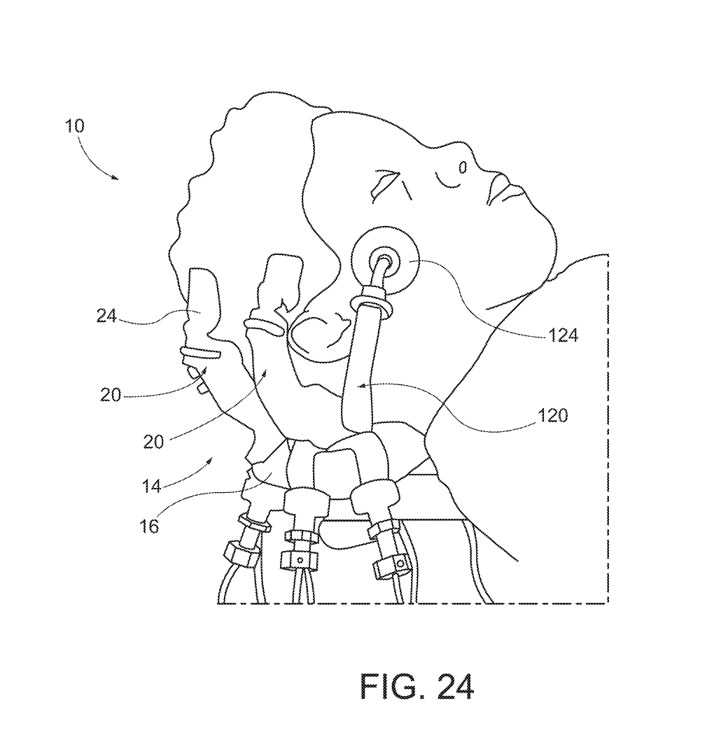

FIG. 24 illustrates another configuration for the HFD (14). With the configuration of the present example of FIG. 24, both types of atraumatic tips (24, 124) are used together, with some clamp arms (20) using atraumatic tip (24) and other clamp arms (20) using atraumatic tip (124). Additionally, the clamp members (20) used in the present example are shorter in length compared to, e.g. the clamp member (20) of FIG. 23. As described above, the number of intermediate links (78) used with the arm (44) may vary to configure the arm (44) to a desired length. Moreover, with any given HFD as described herein, clamp members (20) of any desired length may be used. For instance, the clamp members (20) may all be the same length, may all be different lengths, or may be a combination of some the same length and some different lengths. The various ways to configure the length of the clamp members (20) will be apparent to those of ordinary skill in the art in view of the teachings herein.

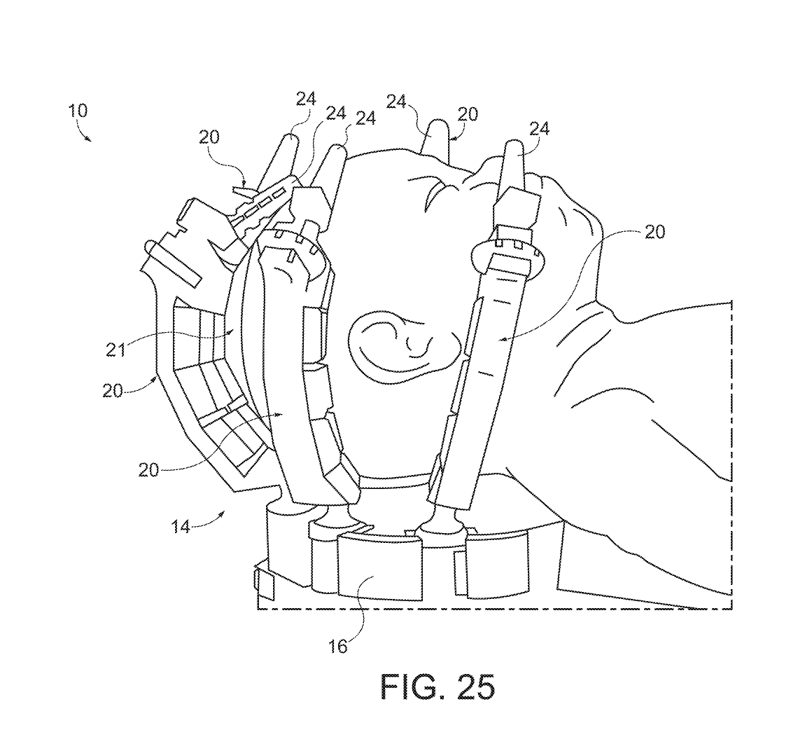

FIG. 25 illustrates another configuration for the HFD (14). In this example, a similar configuration to that shown in FIG. 23 is illustrated. One difference with the present example of FIG. 25 is that the clamp member (20) positioned along the top of the patient's head (11) is configured to provide a point of stabilization through the contact of the atraumatic tip (24) with the head (11) as opposed to the area of stabilization illustrated by the comparable clamp member (20) illustrated in FIG. 23. With the example illustrated in FIG. 25, a space (21) is provided between the arm (44) of the clamp member (20) and the head (11).

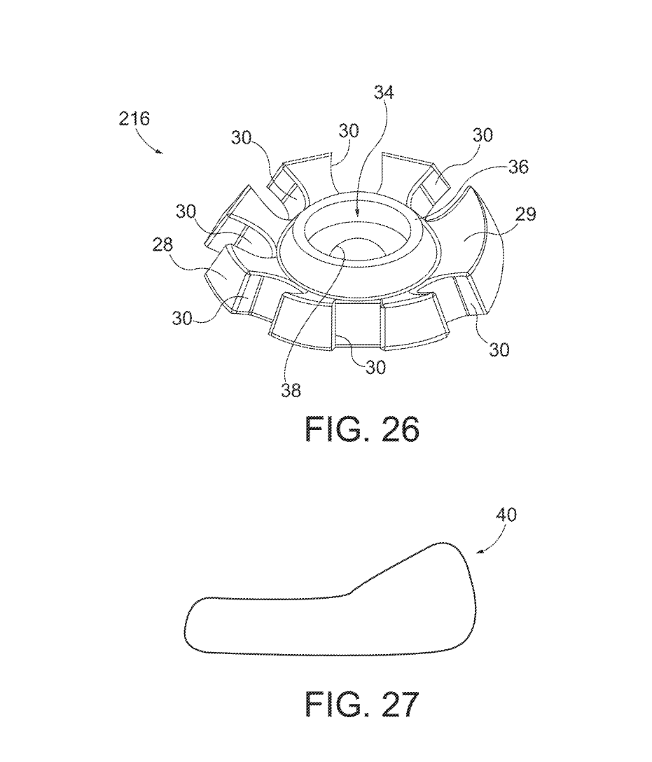

FIG. 26 illustrates an exemplary base (216) that can be used interchangeably with the HFDs described herein. Base (216) is similar to base (16) and base (116) such that the description of bases (16, 116) applies also to base (216). However, the base (216) shows a neck support portion (29). In the present example, the neck support portion (29) is formed as part of the annular flange (28). Also the neck support portion (29) is distally upturned similar to the reminder portions of the flange (28). The neck support portion (29) is configured to receive all or a portion of a head pad (40) to provide additional comfort and support to the patient's neck during stabilization.

FIG. 27 depicts a side elevation view of an exemplary head pad that can be used interchangeably with the HFDs described herein. In the present example, the head pad (40) has a profile where the head pad (40) extends distally a greater degree on one side of the head pad (40). In this manner, that portion of the head pad (40) that extends distally a greater degree may be configured to overlap the neck support portion (29) of the base (216) so as to closely follow the contour of the patient's neck. Accordingly, the head pad (40) in its entirety is configured with a shape that closely matches the contour of the patient's head and neck to provide support and comfort. Other modifications to the head pad (40) to provide comfort and support will be apparent to those of ordinary skill in the art in view of the teachings herein.

It should be understood that any one or more of the teachings, expressions, embodiments, examples, etc. disclosed herein may be combined with any one or more of the other teachings, expressions, embodiments, examples, etc. that are disclosed herein. The teachings, expressions, embodiments, examples, etc. disclosed herein should therefore not be viewed in isolation relative to each other. Various suitable ways in which numerous aspects of the present disclosure may be combined will be readily apparent to those of ordinary skill in the art in view of the teachings disclosed herein. Such modifications and variations are intended to be included within the scope of both the present disclosure and the claims.

Having shown and described various embodiments of the present disclosure, further adaptations of the methods and systems described herein may be accomplished by appropriate modifications by one of ordinary skill in the art without departing from the scope of the present disclosure. Several of such potential modifications have been mentioned, and others will be apparent to those skilled in the art. For instance, examples, embodiments, geometrics, materials, dimensions, ratios, steps, and the like discussed above are illustrative and are not required. Accordingly, the scope of the present disclosure should be considered in terms of the following claims and is understood not to be limited to the details of structure and operation shown and described in the specification and drawings.

1-20. (canceled)

21. A head fixation device for stabilizing a head of a patient, the head fixation device comprising:
a. a base configured to support the head of the patient resting thereon;
b. a first clamp member extending from the base, wherein the first clamp member is adjustable and configured to at least partially conform to the head; and
c. a first actuator operatively connected to the first clamp member, wherein selectively moving the first actuator correspondingly adjusts the first clamp member such that at least a portion of the first clamp member engages the head to at least partially stabilize the head.

22. The head fixation device of claim 21, further comprising:
a. a second clamp member extending from the base, wherein the second clamp member is adjustable and configured to at least partially conform to the head; and
b. a third clamp member extending from the base, wherein the third clamp member is adjustable and configured to at least partially conform to the head.

23. The head fixation device of claim 22, further comprising:
a. a second actuator select operatively connected to the second clamp member, wherein selectively moving the second actuator correspondingly adjusts the second clamp member such that at least a portion of the second clamp member engages the head to at least partially stabilize the head; and
b. a third actuator selectively operatively connected to the third clamp member, wherein selectively moving the third actuator correspondingly adjusts the third clamp member such that at least a portion of the third clamp member engages the head to at least partially stabilize the head.

24. The head fixation device of claim 21, wherein the first clamp member includes a plurality of links, the plurality of links movably connected together in series such that plurality of links is configured to selectively articulate and thereby move the first clamp member.

25. The head fixation device of claim 24, further comprising a cable, wherein each of the plurality of links includes a channel extending therethrough that receives the cable, and wherein tensioning the cable articulates the plurality of links.

26. The head fixation device of claim 21, wherein the first clamp member is mounted relative to the base with at least one degree of relative movement.

27. The head fixation device of claim 26, wherein the first clamp member is mounted relative to the base with three degrees of relative movement.

28. The head fixation device of claim 21, wherein the base comprises a plurality of grooves configured to removably receive the first clamp member therein, and the first clamp member is removably received within one of the plurality of grooves.

29. The head fixation device of claim 28, wherein the base further comprises an annular flange, wherein the plurality of grooves is formed in the annular flange.

30. The head fixation device of claim 29, where the annular flange of the base is upturned distally.

31. The head fixation device of claim 28, wherein the base further comprises a neck support portion.

32. The head fixation device of claim 21, wherein the first clamp member includes a distal atraumatic tip configured to engage the head of the patient to at least partially stabilize the head.

33. The head fixation device of claim 32, wherein the distal atraumatic tip is mounted relative to the first clamp member with three degrees of relative movement.

34. The head fixation device of claim 33, wherein the three degrees of relative movement are pitch, yaw, and roll.

35. A head fixation device for stabilizing a head of a patient, the head fixation device comprising:
a. a base configured to support the head of the patient resting thereon; and
b. one or more clamp members connected with the base, wherein the one or more clamp members provide an area of contact for stabilization.

36. The head fixation device of claim 35, wherein the one or more clamp members further provide a point of contact for stabilization.

37. The head fixation device of claim 35, wherein the area of contact for stabilization extends in a distal direction from the base toward a distal end of the one or more clamp members.

38. The head fixation device of claim 35, wherein the one or more clamp members comprise a plurality of support pads extending along a length of the one or more clamp members, wherein the area of contact for stabilization for a select one of the one or more clamp members is defined by a surface area of contact between the plurality of support pads and the head of the patient.

39. The head fixation device of claim 35, wherein the area of contact for stabilization provided by the one or more clamp members conforms to the head of the patient.

40. A method of stabilizing a head of a patient with a head fixation device having a base that provides support for the head from beneath, one or more clamp members attachable with the base, wherein the one or more clamp members provide lateral support for the head, and one or more actuators connected with the one or more clamp members, wherein the one or more actuators adjust the one or more clamp members, the method comprising:
a. connecting the one or more clamp members with the base; and
b. adjusting the one or more clamp members to provide lateral support to the head, wherein the act of adjusting includes actuating the actuator to position an arm of each of the one or more clamp members to provide an area of stabilization and to position a tip of each of the one or more clamp members to provide a point of stabilization.
